(19) United States
(12) Patent Application Publication
(10) Pub. No.: US 2017/0136021 A1
(43) Pub. Date: May 18, 2017

(54) METHODS AND COMPOSITIONS FOR TREATING ENVELOPED VIRUSES

(71) Applicant: THE BOARD OF TRUSTEES OF THE LELAND STANFORD JUNIOR UNIVERSITY, Palo Alto, CA (US)

(72) Inventors: Shirit Einav, Los Altos Hills, CA (US); Gregory Neveu, Stanford, CA (US); Rina Barouch-Bentov, Stanford, CA (US)

(21) Appl. No.: 14/873,339

(22) Filed: Oct. 2, 2015

Related U.S. Application Data
(60) Provisional application No. 62/059,449, filed on Oct. 3, 2014.

(51) Int. Cl. A61K 31/517; A61K 45/06; A61K 31/404

(52) U.S. Cl. CPC A61K 31/517; A61K 31/404; A61K 45/06

(57) ABSTRACT

Described are compositions and methods for treating an enveloped virus in a patient in need thereof. Exemplary enveloped viruses are those in the family Filoviridae and include Ebola virus.

### CROSS-REFERENCE

This application claims the benefit of U.S. Provisional Application No. 62/059,449 filed Oct. 3, 2014, which application is incorporated herein by reference.

### BACKGROUND

A large part of the world population is at risk for infections with emerging viruses. Changes in the global distribution of diseases, combined with the rise of the middle class in heavily endemic countries, have led to an emerging market opportunity where a therapeutic can be developed within a sustainable pharmaceutical framework.

### SUMMARY OF THE INVENTION

Emerging viral infections include those caused by the families Filoviridae, Togaviridae, Bunyaviridae, Coronaviridae and Arenaviridae, which are significant causes of morbidity and mortality in various parts of the world. One virus of the Filoviridae family, Ebola virus, has had an increasingly large number of outbreaks in recent history, affecting multiple countries and persons around the globe. A 2014 Ebola outbreak was the largest in history, resulting in over 11,000 confirmed deaths. Because of the infectious nature of the disease and high morbidity rates averaging around 83%, there is a need for the development of an effective treatment for Ebola virus.

In one aspect, provided herein is a method of treating Filoviridae, Togaviridae, Bunyaviridae, Coronaviridae, or Arenaviridae, the method comprising administering to a patient in need thereof an effective amount of an agent that inhibits binding of the virus to a host μ (mu) subunit of clathrin adaptor protein complexes. In some embodiments, the μ (mu) subunit is selected from μ subunits of clathrin AP1, AP2, AP3, AP4, and AP5 complexes, and combinations thereof. In some embodiments, the μ (mu) subunit is selected from μ subunits of AP2M1/2, AP1M1/2, AP3M1, AP4M1, and AP5M1, and combinations thereof. In some embodiments, the agent inhibits AAK1, GAK, or a combination thereof. In some embodiments, the agent comprises a tyrosine kinase inhibitor. In some embodiments, the agent comprises erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate, or combination thereof. In some embodiments, the patient is infected with a Filoviridae virus or is susceptible for infection with a Filoviridae virus. In some embodiments, the patient is infected with Ebola virus or is susceptible for infection with Ebola virus. In some embodiments, the agent comprises sunitinib, or a salt or solvate thereof, and erlotinib, or a salt or solvate thereof. In some embodiments, the method further comprises administering to the patient in need thereof an effective amount of an anticancer agent, an antiviral agent, or a combination thereof.

In one aspect, provided herein is a method of treating Filoviridae, Togaviridae, Bunyaviridae, Coronaviridae or Arenaviridae, the method comprising administering to a patient in need thereof an effective amount of a tyrosine kinase inhibitor. In some embodiments, the tyrosine kinase inhibitor comprises an epidermal growth factor receptor inhibitor, a platelet-derived growth factor receptor inhibitor, a vascular endothelial growth factor receptor inhibitor, or a combination thereof. In some embodiments, the tyrosine kinase inhibitor comprises erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate or combination thereof. In some embodiments, the patient is infected with a Filoviridae virus or is susceptible for infection with a Filoviridae virus. In some embodiments, the patient is infected with Ebola virus or is susceptible for infection with Ebola virus. In some embodiments, the tyrosine kinase inhibitor comprises erlotinib, or a salt or solvate thereof, and sunitinib, or a salt or solvate thereof. In some embodiments, the method further comprises administering to the patient in need thereof an effective amount of an anticancer agent, an antiviral agent, or a combination thereof.

In one aspect, provided herein is a composition for treating Filoviridae, Togaviridae, Bunyaviridae, Coronaviridae, or Arenaviridae, the composition comprising sunitinib, or a salt or solvate thereof, and erlotinib, or a salt or solvate thereof. In some embodiments, the composition is for treating Ebola virus. In some embodiments, the composition comprises from about 1 mg to about 1 g of sunitinib, or a salt or solvate thereof, and from about 1 mg to about 1 g of erlotinib, or a salt or solvate thereof.

In one aspect, provided herein is a method of treating a Filoviridae infection in a patient, the method comprising administering to the patient an effective amount of at least one tyrosine kinase inhibitor. In some embodiments, the tyrosine kinase inhibitor is an epidermal growth factor receptor inhibitor. In some embodiments, the tyrosine kinase inhibitor is an inhibitor of a host adaptor protein regulator. Exemplary adaptor protein regulators include AP2-associated protein kinase 1 (AAK1) and cyclin G-associated kinase (GAK). In some embodiments, the at least one tyrosine kinase inhibitor is selected from the group consisting of erlotinib, sunitinib, gefitinib, dasatinib, imatinib, lapatinib, imatinib, sorafenib and combinations thereof. In some embodiments, erlotinib is a GAK inhibitor. In some embodiments, sunitinib is an AAK1 inhibitor. In some embodiments, the patient is infected with Ebola virus. In some embodiments, the tyrosine kinase inhibitor is sunitinib. In some embodiments, the effective amount is from about 1 mg to about 1 g sunitinib. In some embodiments, the effective amount is from about 50 mg to about 300 mg sunitinib. In some embodiments, the tyrosine kinase inhibitor is erlotinib. In some embodiments, the effective amount is from about 1 mg to about 1 g erlotinib. In some embodiments, the effective amount is from about 150 mg to about 900 mg erlotinib. In some embodiments, the patient is administered an effective amount of the at least one protein kinase inhibitor once daily, for a total of 5 days. In some embodiments, the method further comprises administering to the patient an effective amount of a second tyrosine kinase inhibitor. In some embodiments, the method further comprises administering to the patient an effective amount of an anticancer agent.

In one aspect, provided herein is a method of treating a Filoviridae, Togaviridae, Bunyaviridae, Coronaviridae or Arenaviridae infection in a patient, the method comprising administering to the patient an effective amount of at least one tyrosine kinase inhibitor. In some embodiments, the patient is infected with Ebola virus. In some embodiments, the patient is infected with Chikungunya virus. In some embodiments, the patient is infected with Corona virus. In some embodiments, the patient is infected with Lassa virus. In some embodiments, the patient is infected with Marburg virus. In some embodiments, the tyrosine kinase inhibitor is an epidermal growth factor receptor inhibitor. In some embodiments, the tyrosine kinase inhibitor is selected from the group consisting of erlotinib, sunitinib, gefitinib, dasatinib, imatinib, lapatinib, imatinib, sorafenib and combinations thereof. In some embodiments, the tyrosine kinase inhibitor is sunitinib. In some embodiments, the effective amount is from about 1 mg to about 1 g sunitinib. In some embodiments, the effective amount is from about 50 mg to about 300 mg sunitinib. In some embodiments, the tyrosine kinase inhibitor is erlotinib. In some embodiments, the effective amount is from about 1 mg to about 1 g erlotinib. In some embodiments, the effective amount is from about 150 mg to about 900 mg erlotinib. In some embodiments, the patient is administered an effective amount of the at least one protein kinase inhibitor once daily, for a total of 5 days. In some embodiments, the method further comprises administering to the patient an effective amount of a second tyrosine kinase inhibitor. In some embodiments, the method further comprises administering to the patient an effective amount of an anticancer agent.

In one aspect, provided herein is a method of treating a Filoviridae infection in a patient comprising administering to the patient an effective amount of an agent that inhibits binding of a Filoviridae virus to a μ subunit of a host adaptor protein. In some embodiments, the adaptor protein is a clathrin adaptor protein. In some embodiments, the μ subunit is selected from the group consisting of μ subunits of AP1, AP2, AP3, AP4, and AP5 complexes, and combinations thereof. In some embodiments, said μ subunit is selected from the group consisting of AP2M1/2, AP1M1/2, AP3M1, AP4M1, AP5M1 and combinations thereof. In some embodiments, said agent inhibits AAK1 or GAK. In some embodiments, the agent inhibits, directly or indirectly, host protein NUMB and/or GGA. In some embodiments, said agent is selected from the group consisting of erlotinib, sunitinib, gefitinib, dasatinib, imatinib, lapatinib, imatinib, sorafenib and combinations thereof. In some embodiments, the method further comprises administering to the patient an effective amount of a second agent that inhibits binding of a Filoviridae virus to a host μ subunit of a clathrin adaptor protein complex. In some embodiments, the method further comprises administering to the patient an effective amount of a protein kinase inhibitor. In some embodiments, the protein kinase inhibitor is a tyrosine kinase inhibitor. In some embodiments, the agent that inhibits binding is sunitinib. In some embodiments, the effective amount of the agent that inhibits binding is from about 1 mg to about 1 g sunitinib. In some embodiments, the effective amount is from about 50 mg to about 300 mg sunitinib. In some embodiments, the agent that inhibits binding is erlotinib. In some embodiments, the effective amount of the agent that inhibits binding is from about 1 mg to about 1 g erlotinib. In some embodiments, the effective amount is from about 150 mg to about 900 mg erlotinib. In some embodiments, the patient is administered an effective amount of the agent that inhibits binding once daily, for a total of 5 days.

In one aspect, provided herein is a method of treating a Filoviridae infection in a patient comprising administering to the patient an effective amount of an agent that inhibits the phosphorylation of a host adaptor protein. In some embodiments, the agent inhibits binding of a Filoviridae virus to a host adaptor protein or a host cellular receptor protein. In some embodiments, the host adaptor protein is selected from the group consisting of AP1, AP2, AP3, AP4, AP5 and combinations thereof. In some embodiments, a host cellular receptor protein is Neimann-Pick C1 (NPC1). In some embodiments, the agent inhibits, directly or indirectly, internalization of NPC1, wherein the host is infected with Ebola virus. In some embodiments, the agent is selected from the group consisting of erlotinib, sunitinib, gefitinib, dasatinib, imatinib, lapatinib, imatinib, sorafenib and combinations thereof. In some embodiments, the method further comprises administering to the patient an effective amount of a second agent that inhibits the phosphorylation of host adaptor protein. In some embodiments, the agent that inhibits phosphorylation is sunitinib. In some embodiments, the effective amount is from about 1 mg to about 1 g sunitinib. In some embodiments, the effective amount is from about 50 mg to about 300 mg sunitinib. In some embodiments, the agent that inhibits phosphorylation is erlotinib. In some embodiments, the effective amount is from about 1 mg to about 1 g erlotinib. In some embodiments, the effective amount is from about 150 mg to about 900 mg erlotinib.

In one aspect, provided herein is a method of inhibiting infection by a Filoviridae, Togaviridae, Bunyaviridae, Coronaviridae or Arenaviridae, the method comprising administering to a patient in need thereof an effective amount of at least one tyrosine kinase inhibitor. In one aspect, provided herein is a method of inhibiting infection by a Filoviridae, the method comprising administering to a patient in need thereof an effective amount of at least one tyrosine kinase inhibitor.

In one aspect, provided herein is a method of inhibiting infection by a Filoviridae, Togaviridae, Bunyaviridae, Coronaviridae or Arenaviridae, the method comprising administering to a patient in need thereof an effective amount of an agent that inhibits binding of the virus to a host μ subunit of an adaptor protein complex. In one aspect, provided herein is a method of inhibiting infection by a Filoviridae, the method comprising administering to a patient in need thereof an effective amount of an agent that inhibits binding of a Filoviridae virus to a host μ subunit of an adaptor protein complex.

In one aspect, provided herein is a method of reducing Filoviridae, Togaviridae, Bunyaviridae, Coronaviridae or Arenaviridae viral load, the method comprising administering to a patient in need thereof an effective amount of at least one tyrosine kinase inhibitor. In one aspect, provided herein is a method of reducing Filoviridae viral load in a patient in need thereof, the method comprising administering to a patient in need thereof an effective amount of at least one tyrosine kinase inhibitor.

In one aspect, provided herein is a method of reducing Filoviridae, Togaviridae, Bunyaviridae, Coronaviridae or Arenaviridae viral load, the method comprising administering to a patient in need thereof an effective amount of an agent that inhibits binding of a Filoviridae virus to a host μ subunit of an adaptor protein complexes. In one aspect, provided herein is a method of reducing Filoviridae viral load in a patient in need thereof, the method comprising administering to a patient in need thereof an effective amount of an agent that inhibits binding of a Filoviridae virus to a host μ subunit of an adaptor protein complex.

In one aspect, provided herein is a composition comprising from about 1 mg to about 1 g sunitinib and from about 1 mg to about 1 g erlotinib. In some embodiments, the composition comprises from about 50 mg to about 300 mg sunitinib and from about 150 mg to about 900 mg erlotinib. In some embodiments, the composition is configured to treat a Filoviridae infection. In some embodiments, the composition is configured to inhibit infection by a Filoviridae. In some embodiments, the composition is configured to treat a Filoviridae, Togaviridae, Bunyaviridae, Coronaviridae and/or Arenaviridae infection. In some embodiments, the composition is configured to inhibit infection by a Filoviridae, Togaviridae, Bunyaviridae, Coronaviridae or Arenaviridae.

In one aspect, provided herein is a composition for treating a Filoviridae infection, the composition comprising from about 1 mg to about 1 sunitinib and from about 1 mg to about 1 g erlotinib.

In one aspect, provided herein is a composition for treating a Filoviridae infection, the composition comprising from about 50 mg to about 300 mg sunitinib and from about 150 mg to about 900 mg erlotinib.

In one aspect, provided herein is a composition for treating a Filoviridae infection in a patient in need thereof, the composition comprising from about 50 mg to about 300 mg sunitinib and from about 150 mg to about 900 mg erlotinib, wherein the patient in need thereof has been diagnosed with an infection caused by a Filoviridae.

### BRIEF DESCRIPTION OF THE DRAWINGS

The novel and inventive features of the subject matter described herein are set forth with particularity in the appended claims. A better understanding of the feature and advantages of the present invention will be obtained by reference to the following detailed description that sets forth illustrative embodiments, in which the principles of the invention are utilized, and the accompanying drawings of which:

FIG. 1 is a graph showing Ebola viral entry in AAK1 and GAK knockdown cells and NT control cells. The displayed Ebola entry is expressed as a fraction of GFP positive (infected) cells out of a total live cell population. The values are expressed relative to NT control. A representative experiment performed in triplicates is shown. *** indicates a p<0.001 by the Student's t-test.

FIG. 2 is a graph showing inhibition of Ebola virus by erlotinib in a cellular model of infection.

FIG. 3A is a graph of Ebola entry in a cellular model of infection as a function of sunitinib and erlotinib concentration relative to vehicle control.

FIG. 3B is a graph showing a synergistic effect of sunitinib and erlotinib on Ebola viral entry in a cellular model of infection at the 95% confidence interval, as assessed with MacSynergy II software. Peaks above the theoretical additive plane indicate synergy.

FIG. 4A is a graph of Ebola infection and cell viability in a cellular model of infection as a function of sunitinib concentration.

FIG. 4B is a graph of Ebola infection and cell viability in a cellular model of infection as a function of erlotinib concentration.

### DETAILED DESCRIPTION OF THE INVENTION

Disclosed herein, in various aspects, are compositions and methods for treating and inhibiting infection caused by one or more enveloped viruses. Exemplary enveloped viruses include, without limitation, Filoviridae, Togaviridae, Bunyaviridae, Coronaviridae and Arenaviridae. Enveloped viruses comprise a viral envelope having glycoproteins which identify and bind to receptor sites on a host cell membrane, fusing with the host membrane to result in host cell infection. In various embodiments, compositions and methods disclosed herein target host cell receptor sites to interfere with viral binding and host cell infection. In some embodiments, the composition binds to a host cell protein. In some embodiments, the composition inhibits phosphorylation of a host cell protein. In some embodiments, the composition interferes with clathrin mediated viral endocytosis. In some embodiments, the composition comprises a tyrosine kinase inhibitor. In some embodiments, the composition comprises an anticancer agent.

In one aspect of the subject matter provided herein is a method of treating an infection caused by an enveloped virus, including, but not limited to, viral families Filoviridae, Togaviridae, Bunyaviridae, Coronaviridae and Arenaviridae. In some embodiments, the infection is caused by a Filoviridae. In some embodiments, the infection is caused by Ebola virus. In some embodiments, the method comprises administering to a patient in need thereof an effective amount of an agent that inhibits viral binding to a host cell protein. An exemplary host cell protein is a μ subunit of clathrin adaptor protein (AP) complexes, including, for example, a μ subunit of clathrin AP1, AP2, AP3, AP4, or AP5 complexes. In some embodiments, the method comprises administering to a patient in need thereof an effective amount of an agent that inhibits the phosphorylation of a host adaptor protein. In some embodiments, the method comprises administering to a patient in need thereof an effective amount of a tyrosine kinase inhibitor. Examples of tyrosine kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, and salts, solvates and combinations thereof. In some cases, the tyrosine kinase inhibitor is an agent that inhibits viral binding to a host cell protein. In some instances, the method comprises administering to a patient in need thereof a tyrosine kinase inhibitor and an anticancer agent, antiviral agent, or both anticancer and antiviral agents. In some cases, a patient in need thereof is diagnosed as being infected with, or suspected of being infected with, the enveloped virus. In some embodiments, the method results in a reduction in viral load in a patient infected with an enveloped virus.

In one aspect of the subject matter provided herein is a method of inhibiting an infection in a patient by an enveloped virus, including, but not limited to, viral families Filoviridae, Togaviridae, Bunyaviridae, Coronaviridae and Arenaviridae. In some embodiments, the patient has been exposed to, or is suspected of being exposed to, a Filoviridae. In some embodiments, the patient has a likelihood of being exposed to a Filoviridae. In some embodiments, the patient has been exposed to, or is suspected of being exposed to Ebola virus. In some embodiments, the patient has a likelihood of being exposed to Ebola virus. In some embodiments, the method comprises administering to a patient in need thereof an effective amount of an agent that inhibits viral binding to a host cell protein. An exemplary host cell protein is a μ subunit of clathrin adaptor protein complexes, including, for example, a μ subunit of clathrin AP1, AP2, AP3, AP4, or AP5 complexes. In some embodiments, said μ subunit is selected from the group consisting of AP2M1/2, AP1M1/2, AP3M1, AP4M1, AP5M1 and combinations thereof. In some embodiments, the method comprises administering to a patient in need thereof an effective amount of an agent that inhibits the phosphorylation of a host adaptor protein. In some embodiments, the method comprises administering to a patient an effective amount of a tyrosine kinase inhibitor. Examples of tyrosine kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, and salts, solvates and combinations thereof. In some cases, the tyrosine kinase inhibitor is an agent that inhibits viral binding to a host cell protein. In some instances, the method comprises administering to a patient in need thereof a tyrosine kinase inhibitor and an anticancer agent, antiviral agent, or both anticancer and antiviral agents. In some cases, a patient in need thereof is diagnosed as being infected with an enveloped virus, is suspected of being infected with an enveloped virus, or has been exposed to an enveloped virus. In some cases, a patient in need thereof has a likelihood of being exposed to an enveloped virus. In some embodiments, the method results in a reduction in viral load in a patient exposed to an enveloped virus.

In one aspect of the subject matter provided herein is a method of administering a pharmaceutical composition to a patient diagnosed with an infection caused by an enveloped virus. Enveloped viruses include those in the families Filoviridae, Togaviridae, Bunyaviridae, Coronaviridae and Arenaviridae. In exemplary embodiments, the patient is diagnosed with a Filoviridae. In some embodiments, the patient is diagnosed with Ebola virus. In some embodiments, the method comprises administering to a patient in need thereof an effective amount of an agent that inhibits viral binding to a host cell protein. An exemplary host cell protein is a μ subunit of clathrin adaptor protein complexes, including, for example, a μ subunit of clathrin AP1, AP2, AP3, AP4, or AP5 complexes. In some embodiments, said μ subunit is selected from the group consisting of AP2M1/2, AP1M1/2, AP3M1, AP4M1, AP5M1 and combinations thereof. In some embodiments, the method comprises administering to a patient in need thereof an effect amount of an agent that inhibits the phosphorylation of a host adaptor protein. In some embodiments, the method comprises administering to a patient in need thereof an effective amount of a tyrosine kinase inhibitor. Examples of tyrosine kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, and salts, solvates and combinations thereof. In some cases, the tyrosine kinase inhibitor is an agent that inhibits viral binding to a host cell protein. In some instances, the method comprises administering to a patient in need thereof a tyrosine kinase inhibitor and an anticancer agent, antiviral agent, or both anticancer and antiviral agents. In some embodiments, the method results in a reduction in viral load in a patient diagnosed with an infection caused by an enveloped virus.

In one aspect of the subject matter provided herein is a method of monitoring a response of a patient infected with an enveloped virus to treatment with a composition of this disclosure. In some embodiments, the method comprises administering a composition to a patient infected with an enveloped virus and monitoring one or more signs or symptoms of disease caused by the enveloped virus in the patient. Signs and symptoms include, without limitation, headache, fever, muscle pain, vomiting, hemorrhage, hematemesis, diarrhea, diarrhea with blood, abdominal pain, and prostration. In some embodiments, the composition comprises an agent that inhibits viral binding to a host cell protein. In some embodiments, the composition comprises a tyrosine kinase inhibitor. In some cases, the agent that inhibits viral binding to a host cell protein is a tyrosine kinase inhibitor. In some embodiments, the tyrosine kinase inhibitor comprises erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate or combination thereof. In some cases, the composition further comprises an anticancer agent, antiviral agent, or both an anticancer and antiviral agent. In a further aspect, provided herein is a method comprising monitoring a response of a patient infected with an enveloped virus to treatment with a composition of this disclosure, and modifying the administration of the composition based on the response of the patient to the treatment. In some cases, modifying the administration of the composition comprises increasing or decreasing a dosage of one or more active agents of the composition. In some cases, modifying the administration of the composition comprises changing the frequency of administration of an agent of the composition. In some cases modifying the administration of the composition comprises cessation of composition administration.

In one aspect of the subject matter provided herein are compositions comprising a tyrosine kinase inhibitor as an active agent. In some embodiments, the tyrosine kinase inhibitor comprises an epidermal growth factor receptor inhibitor, a platelet-derived growth factor receptor inhibitor, a vascular endothelial growth factor receptor inhibitor, or a combination thereof. Examples of tyrosine kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, and salts, solvates and combinations thereof.

In one aspect of the subject matter provided herein are compositions which inhibit virus binding to a host cell membrane. In another aspect, provided are compositions which inhibit clathrin mediated endocytosis of a virus to a host cell. In another aspect, provided are compositions which inhibit pinocytosis of a virus to a host cell. In some embodiments, a composition comprises a tyrosine kinase inhibitor that inhibits viral binding to a host cell membrane, inhibits clathrin mediated endocytosis of a virus to a host cell, inhibits pinocytosis of a virus to a host cell, or any combination thereof.

### Enveloped Viruses

The methods and compositions described herein are useful for patients infected with, exposed to, and/or at risk of being exposed to an enveloped virus. Enveloped viruses include viruses having viral envelopes covering their capsids. A viral envelope typically comprises a lipoprotein bilayer which functions to mediate binding to a host cell, so that the virus may enter and exploit the host cell for viral replication. Enveloped viruses include any virus in the Filoviridae, Togaviridae, Bunyaviridae, Coronaviridae and Arenaviridae families, including those which infect humans and non-human animals. The polynucleotide and polypeptide sequences encoding these viruses are well known in the art and may be found at NCBI's GenBank database, the contents of which database entries are incorporated by reference herein in their entirety. The family Filoviridae includes the genera Marburg virus, Cuevavirus and Ebola virus. Ebola virus species include includes Bundibugyo virus, Reston virus, Sudan virus, Zaire Ebola virus and TaïForest virus. The family Togaviridae includes the genera Alpha virus and Rubi virus. Alpha virus includes Barmah Forest virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus, and Western equine encephalitis virus. The family Bunyaviridae includes the genera Hantavirus, Nairovirus, Orthobunyavirus, Phlebovirus and Tospovirus. The family Coronaviridae includes the genera Alphacoronavirus, Betacoronavirus, Deltacoronavirus, Gammacoronavirus, Bafinivirus and Torovirus. The family Arenaviridae includes the genus Arenavirus. Arenaviruses include Lassa virus, Lymphocytic choriomeningitis virus, Junin virus, Machupo virus, Guanarito virus, Sabia virus, Chapare virus, and Lujo virus.

Without being bound by theory, during infection by some enveloped viruses, viral proteins hijack host adaptor protein complexes to mediate viral particle traffic at temporally distinct steps of the viral lifecycle, such as viral entry, assembly, release and cell to cell spread. In one aspect, the compositions and methods described herein treat viral infection from an enveloped virus by inhibiting host-virus interactions. In some embodiments, the host-virus interaction is inhibited by administering to a patient in need thereof an agent that targets one or more host cell proteins exploited by an enveloped virus during infection. In some embodiments, the target host cell protein is a protein that modulates the activity of μ subunits of host clathrin adaptor protein complexes. Examples of host clathrin adaptor proteins include, without limitation, AP2M1/2, AP1M1/2, AP3M1, AP4M1 and AP5M1. In some embodiments, the host clathrin adaptor protein is AP2M1. In some embodiments, the host cell protein that modulates the activity of μ subunits of clathrin adaptor protein complexes (i.e. a clathrin adaptor protein regulator) is a protein kinase such as AP2-associated protein kinase 1 (AAK1) and/or cyclin G-associated kinase (GAK). In some embodiments, host proteins NUMB and/or GGA (Golgi-localising, Gamma-adaptin ear domain homology, ARF-binding proteins) are host cell protein targets. In some cases, inhibition of host-virus interaction is accomplished by inhibiting phosphorylation of NUMB by AAK1. In some cases, the administration of a protein kinase inhibitor is useful in abolishing the interaction of μ subunits of clathrin adaptor protein complexes with an enveloped virus. In some embodiments, administration of a protein kinase inhibitor to a patient in need thereof inhibits or attenuates host cell viral infection by an enveloped virus. In various embodiments, provided herein are kinase inhibitors which inhibit or attenuate host cell-virus interaction, wherein the virus includes, without limitation, enveloped viruses of the families Filoviridae (e.g., Ebola virus), Togaviridae, Bunyaviridae, Coronaviridae and Arenaviridae. In exemplary embodiments, a kinase inhibitor is useful for the inhibition and/or treatment of a host infected with Ebola virus. In some embodiments, a kinase inhibitor is useful for the inhibition and/or treatment of a host infected with a clathrin adaptor protein binding virus. In some embodiments, a protein kinase inhibitor comprises erlotinib, sunitinib, gefitinib, dasatinib, imatinib, lapatinib, imatinib, sorafenib, or a combination thereof. In some cases, a kinase inhibitor comprises erlotinib and sunitinib.

An aspect of this disclosure provides protein kinase inhibitors that have efficacy against enveloped viruses, such as Ebola virus, that enter a target cell via clathrin-mediated endocytosis. The entry of Ebola virus into a host cell is dependent on AP2 and a tyrosine motif within the Ebola receptor, Niemann-Pick C1. Experiments performed by the inventors demonstrate that the tyrosine kinase inhibitor erlotinib reduces Ebola replication in cells as well as mortality of Ebola infected mice. The inventors have also identified that pharmacological inhibition of AAK1 and/or GAK is useful as an antiviral against Ebola virus. In particular, tyrosine kinase inhibitors, in many implementations, target host cell entry, viral assembly, and/or viral release. In some embodiments, AAK1 and GAK are validated as anti-Ebola targets using non-pathogenic virus-like particles. Administration of a tyrosine kinase inhibitor to patients infected with an enveloped virus (e.g., Ebola virus), in many instances, is useful for the treatment or the prevention of severe complications and/or mortality. Administration of a tyrosine kinase inhibitor to subjects, in many instances, is useful as prophylactic prevention of community outbreaks. Administration of a tyrosine kinase inhibitor to travelers to endemic countries or military personnel, in many instances, is useful as prophylactic prevention of infection. Examples of tyrosine kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, and salts, solvates and combinations thereof. In some embodiments, provided herein is a composition comprising one or more tyrosine kinase inhibitors as active agents for the treatment of infection by an enveloped virus. In some embodiments, the active agent is erlotinib, or a salt or solvate thereof. In some embodiments, the active agent is sunitinib, or a salt or solvate thereof. In some embodiments, the composition comprises erlotinib, or a salt or solvate thereof, and sunitinib, or a salt or solvate thereof. In some embodiments, the composition further comprises an anticancer agent, an antiviral agent, or a combination thereof.

### Methods of Treatment

Provided herein, in various embodiments, are methods of treating a patient infected with, exposed to, or susceptible of being exposed to an enveloped virus, the methods comprise administering to the patient a composition comprising an agent that inhibits binding of the enveloped virus to a host protein. In some cases, the host protein is a μ subunit of an adaptor protein (AP) complex. In some cases, the agent is a tyrosine kinase inhibitor. In some embodiments, the patient is co-infected with one or more additional viruses.

Compositions and methods described herein are useful for the treatment of enveloped viruses in a subject (i.e. patient, host), which includes, without limitation, humans and non-human mammals (e.g., mice, rats, pigs, cats, dogs, horses). Typical subjects to which agents of the present disclosure may be administered are mammals, particularly primates, including humans. Additional subjects include, without limitation, livestock such as cattle, sheep, goats, and swine; poultry such as chickens, ducks, geese, and turkeys; and domesticated animals such as dogs and cats. In addition, subjects include, without limitation, subjects suitable for diagnostic or research applications. Additional subjects include, without limitation, rodents such as mice, rats and hamsters; rabbits; primates and swine such as inbred pigs. The terms do not denote a particular age. Thus, both adult and newborn subjects are intended to be covered.

In many instances, a treatment is an act upon a disease, disorder or condition with an agent to reduce or ameliorate the pharmacologic and/or physiologic effects of the disease, disorder or condition and/or its symptoms. As used herein, a disease is caused by an enveloped virus of the Filoviridae, Togaviridae, Bunyaviridae, Coronaviridae and/or Arenaviridae family. In some embodiments, treatment includes reducing the risk of occurrence of a disease in a subject determined to be predisposed to the disease but not yet diagnosed as infected with the disease. In this instance, a treatment may be considered a way of inhibiting infection by said disease. In some instances, a treatment which inhibits infection of a disease is a vaccine. In some embodiments, treatment includes impeding the development of a disease. In some embodiments, treatment includes relieving a disease by causing regression of the disease and/or relief from one or more disease symptoms. Treatment, in many implementations, includes the delivery of an agent to provide a pharmacologic effect, even in the absence of a disease or condition. For example, treatment may encompass delivery of a disease or viral inhibiting agent that provides for enhanced or desirable effects in a subject. Examples of effects include, without limitation, reduction of viral load and a reduction of disease symptoms. In various embodiments, treatment comprises administration of a composition comprising an active agent. In some embodiments, a tyrosine kinase inhibitor is an active agent of a composition described herein. In some embodiments, an anticancer agent is an active agent of a composition described herein.

In some embodiments, a method of treating an enveloped virus comprises administering to a patient in need thereof an effective amount of an agent that inhibits binding of the enveloped virus to a host protein. In some embodiments, the patient is infected with, at risk of being infected with, or suspected of being infected with, Ebola virus. In some embodiments, the agent comprises erlotinib or a salt or solvate thereof. In some embodiments, the agent comprises sunitinib or a salt or solvate thereof. In some embodiments, the agent comprises erlotinib or a salt or solvate thereof, and sunitinib or a salt or solvate thereof. In some cases, the method further comprises administering to the patient an anticancer agent. In some cases, the method further comprises administering to the patient an antiviral agent.

In some embodiments, a method of treating an enveloped virus comprises administering to a patient in need thereof an effective amount of an agent that inhibits phosphorylation of a host protein. In some embodiments, the patient is infected with, at risk of being infected with, or suspected of being infected with, Ebola virus. In some embodiments, the agent comprises erlotinib or a salt or solvate thereof. In some embodiments, the agent comprises sunitinib or a salt or solvate thereof. In some embodiments, the agent comprises erlotinib or a salt or solvate thereof, and sunitinib or a salt or solvate thereof. In some cases, the method further comprises administering to the patient an anticancer agent. In some cases, the method further comprises administering to the patient an antiviral agent.

In some embodiments, a method of treating an enveloped virus comprises administering to a patient in need thereof an effective amount of a tyrosine kinase inhibitor. In some embodiments, the tyrosine kinase inhibitor comprises an epidermal growth factor receptor inhibitor, a platelet-derived growth factor receptor inhibitor, a vascular endothelial growth factor receptor inhibitor, or a combination thereof. In some embodiments, the patient is infected with, at risk of being infected with, or is suspected of being infected with, Ebola virus. In some embodiments, the agent comprises erlotinib or a salt or solvate thereof. In some embodiments, the agent comprises sunitinib or a salt or solvate thereof. In some embodiments, the agent comprises erlotinib or a salt or solvate thereof, and sunitinib or a salt or solvate thereof. In some cases, the method further comprises administering to the patient an anticancer agent. In some cases, the method further comprises administering to the patient an antiviral agent.

In some embodiments, a method of treating an enveloped virus comprises administering to a patient in need thereof an effective amount of erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate or combination thereof. In some embodiments, the patient is infected with, at risk of being infected with, or is suspected of being infected with, Ebola virus. In some cases, the method further comprises administering to the patient an anticancer agent. In some cases, the method further comprises administering to the patient an antiviral agent. In some cases, a method of treating an enveloped virus comprises administering to a patient in need thereof an effective amount of erlotinib, or a salt or solvate thereof. In some cases, a method of treating an enveloped virus comprises administering to a patient in need thereof an effective amount of sunitinib, or a salt or solvate thereof. In some cases, a method of treating an enveloped virus comprises administering to a patient in need thereof an effective amount of erlotinib, or a salt or solvate thereof, and sunitinib, or a salt or solvate thereof.

In one aspect, provided herein are treatments comprising the administration of one or more compositions as described herein (e.g., tyrosine kinase inhibitor) and one or more additional therapies. Additional therapies include antiviral therapies. In some embodiments, an additional therapy includes side effect therapy. Additional treatment options include, but are not limiting to, fluid replacement therapy and an analgesic (e.g., acetaminophen).

In some embodiments, a diagnosis method is utilized at one or more time point during and/or after treatment to monitor viral load. In some embodiments, the treatment regimen is adjusted according to the diagnostic results.

In some embodiments, a subject having viral infection (e.g., Ebola virus) is evaluated using a scoring matrix. The scoring matrix may include criteria including platelet counts, white blood cell count, Hematocit/Hb, blood pressure, and fever values. The scoring matrix may be used to determine a method of treatment, for example, active agent selection, dosage, treatment duration, among others.

### Pharmaceutical Compositions

Provided herein, in various aspects, are compositions comprising one or more active agents. In some embodiments, the compositions comprise therapeutically effective amounts of one or more active agents to treat a patient infected with an enveloped virus. In some embodiments, a patient infected with an enveloped virus has been diagnosed as being infected with an enveloped virus. In some embodiments, a patient is suspected of being infected with an enveloped virus. In some embodiments, a composition comprises a therapeutically effective amount of one or more active agents to treat a patient who is at risk of becoming infected with an enveloped virus. Enveloped viruses include, without limitation, those in the families Filoviridae, Togaviridae, Bunyaviridae, Coronaviridae and Arenaviridae. In some embodiments, a patient administered with a composition described herein is infected with Ebola virus. In some embodiments, a patient administered with a composition described herein is diagnosed as having Ebola virus. In some embodiments, a patient administered with a composition described herein is susceptible to infection with Ebola virus.

A pharmaceutical composition, in various embodiments, comprises an active agent as described herein. Active agents include, without limitation, tyrosine kinase inhibitors, antiviral agents and anticancer agents. In some embodiments, an active agent comprises an agent that inhibits binding of a virus to a host cell protein. In some embodiments, an active agent comprises a tyrosine kinase inhibitor. In some embodiments, a pharmaceutical composition is suitable for administration to a subject, such as a human. In some embodiments, the composition is sterile and preferably free of contaminants that are capable of eliciting an undesirable response within a subject. In some embodiments, the composition is of pharmaceutical grade.

In one aspect, provided herein are compositions comprising one or more kinase inhibitors. A kinase inhibitor includes a tyrosine kinase inhibitor. In some embodiments, a composition comprises 1, 2, 3, 4, 5 or more kinase inhibitors. In some implementations, active agents of a composition are administered together or separately. Examples of kinase inhibitors include, without limitation, erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate or combination thereof. In some embodiments, an active agent comprises sunitinib. In some embodiments, an active agent comprises erlotinib. In some embodiments, a composition comprises erlotinib. In some embodiments, a composition comprises sunitinib. In some embodiments, a composition comprises erlotinib and sunitinib. In some embodiments, a patient infected with an enveloped virus, a patient suspected to be infected with an enveloped virus or a patient susceptible to infection with an enveloped virus is administered a composition comprising one or more tyrosine kinase inhibitors. In some embodiments, the patient is administered erlotinib. In some embodiments, the patient is administered sunitinib. In some embodiments, the patient is administered both erlotinib and sunitinib. In some embodiments, the patient is administered a dose of erlotinib and a dose or sunitinib at separate times. In some embodiments, the composition further comprises an anticancer agent. In some embodiments, the composition further comprises an antiviral agent.

In some embodiments, provided herein are compositions comprising an agent that inhibits binding of an enveloped virus to a host μ subunit of adaptor protein (AP) complexes. In some embodiments, the agent comprises a tyrosine kinase inhibitor. In some embodiments, the agent comprises erlotinib, sunitinib, or a salt, solvate, or combination thereof. In some embodiments, the composition further comprises an antiviral agent. In some embodiments, the composition further comprises an anticancer agent.

Examples of antiviral agents useful in the compositions and methods described herein include, without limitation, BCX4430, Brincidofovir, DZNep, Favipiravir, FGI-103, FGI-104, FGI-106, JK-05, Lamivudine, TKM-Ebola, Triazavirin, ZMapp, and vaccines such as cAd3-ZEBOV and VSV-EBOV.

Examples of anticancer agents useful in the compositions and methods described herein include, without limitation, abiraterone; abarelix; abraxane, adriamycin; actinomycin; acivicin; aclarubicin; acodazole hydrochloride; acronine; adozelesin; aldesleukin; alemtuzumab; allopurinol; alitretinoin; altretamine; ametantrone acetate; aminoglutethimide; aminolevulinic acid; amifostine; amsacrine; anastrozole; anthramycin; aprepitant; arsenic trioxide; asparaginase; asperlin; azacitidine; azetepa; azotomycin; batimastat; bendamustine hydrochloride; benzodepa; bevacizumab; bexarotene; bicalutamide; bisantrene hydrochloride; bisnafide dimesylate; bizelesin; bleomycin; bleomycin sulfate; bortezomib; brequinar sodium; bropirimine; busulfan; cactinomycin; calusterone; caracemide; carbetimer; carboplatin; carmustine; carubicin hydrochloride; carzelesin; capecitabine; cedefingol; cetuximab; chlorambucil; cirolemycin; cisplatin; cladribine; clofarabine; crisnatol mesylate; cyclophosphamide; cytarabine; dacarbazine; dasatinib; daunorubicin hydrochloride; dactinomycin; darbepoetin alfa; decitabine; degarelix; denileukin diftitox; dexormaplatin; dexrazoxane hydrochloride; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; eltrombopag olamine; enloplatin; enpromate; epipropidine; epirubicin hydrochloride; epoetin alfa; erbulozole; erlotinib hydrochloride; esorubicin hydrochloride; estramustine; estramustine phosphate sodium; etanidazole; etoposide; etoposide phosphate; etoprine; everolimus; exemestane; fadrozole hydrochloride; fazarabine; fenretinide; filgrastim; floxuridine; fludarabine phosphate; fluorouracil; flurocitabine; fosquidone; fostriecin sodium; fulvestrant; gefitinib; gemcitabine; gemcitabine hydrochloride; gemcitabine—cisplatin; gemtuzumab ozogamicin; goserelin acetate; histrelin acetate; hydroxyurea; idarubicin hydrochloride; ifosfamide; iimofosine; ibritumomab tiuxetan; idarubicin; ifosfamide; imatinib mesylate; imiquimod; interleukin Il (including recombinant interleukin II, or r1L2), interferon alfa-2a; interferon alfa-2b; interferon alfa-n1; interferon alfa-n3; interferon beta-1 a; interferon gamma-1 b; iproplatin; irinotecan hydrochloride; ixabepilone; lanreotide acetate; lapatinib; lenalidomide; letrozole; leuprolide acetate; leucovorin calcium; leuprolide acetate; levamisole; liposomal cytarabine; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; methoxsalen; metoprine; meturedepa; mitindomide; mitocarcin; mitocromin; mitogillin; mitomalcin; mitomycin C; mitosper; mitotane; mitoxantrone hydrochloride; mycophenolic acid; nandrolone phenpropionate; nelarabine; nilotinib; nocodazoie; nofetumomab; nogalamycin; ofatumumab; oprelvekin; ormaplatin; oxaliplatin;oxisuran; paclitaxel; palifermin; palonosetron hydrochloride; pamidronate; pegfilgrastim; pemetrexed disodium; pentostatin; panitumumab; pazopanib hydrochloride; pemetrexed disodium; plerixafor; pralatrexate; pegaspargase; peliomycin; pentamustine; peplomycin sulfate; perfosfamide; pipobroman; piposulfan; piroxantrone hydrochloride; plicamycin; plomestane; pomalidomide, porfimer sodium; porfiromycin; prednimustine; procarbazine hydrochloride; puromycin; puromycin hydrochloride; pyrazofurin; quinacrine; raloxifene hydrochloride; rasburicase; recombinant HPV bivalent vaccine; recombinant HPV quadrivalent vaccine; riboprine; rogletimide; rituximab; romidepsin; romiplostim; safingol; safingol hydrochloride; sargramostim; semustine; simtrazene; sipuleucel-T; sorafenib; sparfosate sodium; sparsomycin; spirogermanium hydrochloride; spiromustine; spiroplatin; streptonigrin; streptozocin; sulofenur; sunitinib malate; talisomycin; tamoxifen citrate; tecogalan sodium; tegafur; teloxantrone hydrochloride; temozolomide; temoporfin; temsirolimus; teniposide; teroxirone; testolactone; thalidomide; thiamiprine; thioguanine; thiotepa; tiazofurin; tirapazamine; topotecan hydrochloride; toremifene; tositumomab and I ¹³¹ Iodine tositumomab; trastuzumab; trestolone acetate; tretinoin; triciribine phosphate; trimetrexate; trimetrexate glucuronate; triptorelin; tubulozole hydrochloride; uracil mustard; uredepa; valrubicin; vapreotide; verteporfin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorinostat; vorozole; zeniplatin; zinostatin; zoledronic acid; and zorubicin hydrochloride.

In some embodiments, an active agent is administered with a NSAID. NSAIDs include, but are not limited to, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, and COX-2 specific inhibitors (such as, but not limited to, celecoxib, rofecoxib, valdecoxib, parecoxib, etoricoxib, lumiracoxib, CS-502, JTE-522, L-745,337 and NS398).

Pharmaceutical compositions are designed for administration to a patient in need thereof via a number of non-limiting routes including, without limitation, oral, intravenous, buccal, rectal, parenteral, intraperitoneal, intradermal, intracheal, intramuscular, subcutaneous and inhalational. The terms administer, administering, administration, and the like, as used herein, refer to the methods that may be used to enable delivery of an agent to the desired site of biological action. These methods include, but are not limited to oral routes, inhalational, transdermal, transmucosal, sublingual, buccal, intraduodenal routes, parenteral injection (including intravenous, subcutaneous, intraperitoneal, intramuscular, intravascular, intraarterial, intracardial, intradermal, intraduodenal, intramedullary, intraosseous, intrathecal, intravitreal, epidural or infusion), topical (including epicutaneous, dermal, enema, eye drops, ear drops, intranasal, vaginal) and rectal administration. In some embodiments, an agent described herein is administered orally. The terms co-administration or the like, as used herein, encompass administration of agents to a single patient, and include treatment regimens in which the agents are administered by the same or different route of administration, at the same or different time. In some embodiments, the agents described herein are administered either alone or in combination with pharmaceutically acceptable carriers, excipients or diluents in a pharmaceutical composition.

In some embodiments, pharmaceutical compositions suitable for oral administration are presented as discrete units such as capsules, cachets or tablets each containing a predetermined amount of an active agent as a powder or granules; as a solution or a suspension in an aqueous liquid or a non-aqueous liquid; or as an oil-in-water liquid emulsion or a water-in-oil liquid emulsion. In some embodiments, the active agent is presented as a bolus, electuary or paste.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, optionally with an added preservative. The compositions may take such forms as suspensions, solutions or emulsions in oily or aqueous vehicles, and may contain formulatory agents such as suspending, stabilizing and/or dispersing agents. In some embodiments, the compositions are formulated in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Pharmaceutical compositions for parenteral administration include aqueous and non-aqueous (oily) sterile injection solutions of the active agents which may contain antioxidants, buffers, bacteriostats and solutes which render the formulation isotonic with the blood of the intended recipient; and aqueous and non-aqueous sterile suspensions which may include suspending agents and thickening agents.

In some embodiments, pharmaceutical compositions are formulated as a depot preparation, for example, for administration by implantation (e.g., subcutaneously or intramuscularly) or by intramuscular injection. In some cases, the compositions are formulated with suitable polymeric or hydrophobic materials or ion exchange resins, or as sparingly soluble derivatives, for example, as a sparingly soluble salt.

In some embodiments, pharmaceutical compositions are formed into tablets, lozenges, pastilles or gels for buccal or sublingual administration.

In some embodiments, pharmaceutical compositions are formulated in rectal compositions such as suppositories or retention enemas, e.g., containing conventional suppository bases such as cocoa butter, polyethylene glycol, or other glycerides.

In some embodiments, pharmaceutical compositions are administered topically such that the compound does not significantly enter the blood stream. Pharmaceutical compositions suitable for topical administration include liquid or semi-liquid preparations suitable for penetration through the skin such as gels, liniments, lotions, creams, ointments or pastes, and drops suitable for administration to the eye, ear or nose.

In some embodiments, pharmaceutical compositions are formulated for administration by inhalation using an insufflator, nebulizer pressurized packs or other convenient means of delivering an aerosol spray. Pressurized packs include those comprising a suitable propellant such as dichlorodifluoromethane, trichlorofluoromethane, dichlorotetrafluoroethane, carbon dioxide or other suitable gas. In some cases, pharmaceutical compositions are formulated as a dry powder for administered with the aid of an inhalator or insufflator.

In various aspects, one or more active agents of compositions described herein are in the form of pharmaceutically acceptable salts. In additional embodiments, one or more active agents exist in unsolvated or solvated forms with pharmaceutically acceptable solvents such as water, ethanol, and the like. Reference to an agent that inhibits binding of a virus to a host cell protein herein includes both pharmaceutically acceptable salts and solvates of the agent that inhibits binding of a virus to a host cell protein. Reference to a tyrosine kinase inhibitor herein includes both pharmaceutically acceptable salts and solvates of the tyrosine kinase inhibitor. In some embodiments, pharmaceutically acceptable refers to a material, such as a carrier or diluent, which does not abrogate the biological activity or properties of the agent, and is relatively nontoxic, i.e. the material is administered to a subject without causing undesirable biological effects or interacting in a deleterious manner with any of the agents of the composition in which it is contained.

In some embodiments, a pharmaceutically acceptable salt or “salt” refers to a form of a therapeutically active agent that consists of a cationic form of the therapeutically active agent in combination with a suitable anion, or in alternative embodiments, an anionic form of the therapeutically active agent in combination with a suitable cation. In some embodiments, pharmaceutically acceptable salts are obtained by reacting an active agent with an acid. In some embodiments, the active agent is basic and is reacted with an organic acid or an inorganic acid. In some embodiments, an active agent is prepared as a chloride salt, sulfate salt, bromide salt, mesylate salt, maleate salt, citrate salt or phosphate salt. In some embodiments, an active agent is prepared as a hydrochloride salt. In some embodiments, pharmaceutically acceptable salts are obtained by reacting an active agent with a base. In some embodiments, the active agent is acidic and is reacted with a base. In some embodiments, the active agent is prepared as a sodium salt, calcium salt, potassium salt, magnesium salt, meglumine salt, N-methylglucamine salt or ammonium salt. In some embodiments, the compounds provided herein are prepared as a sodium salt.

In some embodiments, compositions described herein comprise one or more active agents that are prepared as prodrugs. A prodrug refers to an agent that is converted into the parent drug in vivo. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound. In some aspects, provided are compositions comprising a prodrug formulation of an agent that inhibits binding of a virus to a host cell protein described herein. In some aspects, provided are compositions comprising a prodrug formulation of a tyrosine kinase inhibitor described herein. In some embodiments, an active agent is metabolized upon administration to a subject to produce a metabolite that is then used to produce a desired effect, including a desired therapeutic effect. In some embodiments, a metabolite of an active agent is a derivative of that compound that is formed when the compound is metabolized. The term active metabolite refers to a biologically active derivative of a compound that is formed when the compound is metabolized. In some cases, a prodrug is easier to administer than the parent drug, for example, the prodrug is bioavailable by oral administration whereas the parent is not. In some cases, the prodrug has improved solubility in pharmaceutical compositions over the parent drug. In a non-limiting example, a prodrug of an active agent described herein is administered as an ester prodrug to facilitate transmittal across a cell membrane where water solubility is detrimental to mobility, where the prodrug is metabolically hydrolyzed to the carboxylic acid, the active entity, once inside the cell where water-solubility is beneficial. As a further non-liming example, a prodrug is a short peptide bonded to an acid group, where the peptide is metabolized to reveal the active moiety. In some embodiments, a prodrug is designed to alter the metabolic stability and/or the transport characteristics of an active agent, to mask side effects and/or toxicity, to improve the flavor of an agent, and/or to alter other characteristics or properties of the active. In some embodiments, some of the herein-described compounds are prodrugs for other derivatives or active compounds. In some embodiments, some of the herein-described active agents are formulated as prodrugs.

Further provided herein are pharmaceutical combinations that result from the mixing or combining of more than one active agent. A pharmaceutical combination includes both fixed and non-fixed combinations of the active agents. In some cases, the active agents are provided in a fixed combination, where the active agents of the fixed combination are administered to a patient simultaneously in the form of a single entity or dosage. In some cases, the active agents are provided in a non-fixed combination, wherein the active agents of the non-fixed combination are administered to a patient as separate entities either simultaneously, concurrently or sequentially with no specific intervening time limits, and wherein such administration provides effective levels of the two compounds in the body of the patient. The latter also applies to cocktail therapy, for example, the administration of three or more active agents.

A therapeutically effective amount of an agent or composition is generally the amount of an agent or composition that is required to relieve to some extent one or more symptoms of a disease being treated (i.e. viral infection) and/or the amount that will prevent, to some extent, one or more symptoms of a disease that the host being treated has or is at risk of developing. The terms effective amount or therapeutically effective amount, as used herein, refer to a sufficient amount of an agent or composition being administered, which will relieve to some extent one or more of the symptoms of the disease being treated. The result includes reduction and/or alleviation of the signs, symptoms, or causes of a disease, or any other desired alteration of a biological system. For example, an effective amount for therapeutic uses is the amount of the composition comprising a compound as disclosed herein required to provide a clinically significant decrease in disease symptoms. An appropriate effective amount in any individual case is optionally determined using techniques, such as a dose escalation study.

A unit dosage form, in many instances, refers to physically discrete units suitable as unitary dosages for subjects, wherein each unit comprises a predetermined quantity of a composition comprising an active agent such as a tyrosine kinase inhibitor or anticancer agent, as described herein. In many embodiments, a composition further comprises a pharmaceutically acceptable diluent, carrier or vehicle. The specifications for unit dosage forms depend on the particular composition employed, the route and frequency of administration, the effect to be achieved and the pharmacodynamics associated with the composition in the host.

It is to be understood that the terminology used herein is for the purpose of describing particular embodiments only, and is not intended to be limiting.

### Pharmaceutical Formulations

In one aspect, provided herein are compositions comprising one or more active agents suitable for inhibiting viral interaction with a host cell or a component of a host cell, such as a host protein. Host proteins include adaptor proteins and regulators of adaptor proteins. In some embodiments, the adaptor proteins are clathrin adaptor proteins. In some embodiments, the active agent is a tyrosine kinase inhibitor. Examples of clathrin adaptor protein regulators include, without limitation, AAK1 and GAK. Inhibitors of GAK and AAK1 include, but are not limited to, RNAi, antisense, ribozymes or small molecules that bind to GAK or AAK1. Additional active agents include antibodies configured to bind to clathrin adaptor proteins and/or regulators of clathrin adaptor proteins. In addition, dominant-negative binding proteins or aptamers can inhibit AAK1 or GAK. In some embodiments, decoy receptors or polypeptides corresponding to binding sites from AP2M1 or other μ subunits of clathrin AP complexes can inhibit GAK or AAK1.

Embodiments of the compositions include salts and prodrugs of the active agent. In some embodiments, the composition is a liquid, gel or solid. In some embodiments, the composition is in the form of a controlled release or a sustained release formulation.

Provided herein are compositions comprising one or more active agents, for example, a tyrosine kinase inhibitor, formulated with one or more pharmaceutically acceptable excipients, diluents, carriers and/or adjuvants. In addition, compositions of the disclosure include active agents formulated with one or more pharmaceutically acceptable auxiliary substances. Auxiliary substances, such as pH adjusting and buffering agents, tonicity adjusting agents, stabilizers, wetting agents and the like are readily available to the public. Suitable excipient vehicles for a composition include, for example, water, saline, dextrose, glycerol, ethanol and combinations thereof. In addition, the vehicle may comprise auxiliary substances such as wetting or emulsifying agents or pH buffering agents.

An active agent of a composition described herein is administered to a patient using any means capable to result in a desired effect. For example, a decrease in one or more symptoms, a decrease in viral load, reduction in symptoms cause by viral infection, and/or inhibition of viral infection. In many embodiments, the active agent is formulated into a pharmaceutical composition by combination with appropriate, pharmaceutically acceptable carriers or diluents, into solid, semi-solid, liquid or gaseous forms, such as tablets, capsules, powders, granules, ointments, solutions, suppositories, injections, inhalants and aerosols. For oral preparations, the active agent may be used alone or in combination with appropriate additives to make tablets, powders, granules or capsules, for example, with conventional additives, such as lactose, mannitol, corn starch, or potato starch; with binders such as crystalline cellulose, cellulose derivatives, acacia, corn starch or gelatins; with disintegrators, such as corn starch, potato starch, or sodium carboxymethylcellulose; with lubricants, such as talc or magnesium stearate; and/or if desired, with diluents, buffering agents, moistening agents, preservatives or flavoring agents.

Compositions described herein, in various implementations, comprise a sustained-release or controlled release matrix. In addition, embodiments of the compositions may be used in conjunction with other treatments that use sustained-release formulations. A sustained-release matrix, in many instances, is a matrix made of materials, usually polymers, which are degradable by enzymatic or acid-based hydrolysis or by dissolution. Once inserted into the body, the matrix may be acted upon by enzymes and body fluids. Examples of sustained-release matrix materials include, without limitation, liposomes, polylactides (polylactic acid), polyglycolide (polymer of glycolic acid), polylactide co-glylide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids (e.g., phenylalanine, tyrosine, isoleucine), polynucleotides, polyvinyl propylene, polyvinylpyrrolidone and silicone. Illustrative biodegradable matrices include a polylactide matrix, a polyglycolide matrix and a polylactide co-glycolide (co-polymers of lactic acid and glycolic acid) matrix.

In some embodiments, an active agent is formulated into a preparation for injection by dissolving, suspending or emulsifying the agent in an aqueous or non-aqueous solvent, such as vegetable or other similar oils, synthetic aliphatic acid glycerides, esters of high aliphatic acids, or propylene glycol; and if desired, with conventional additives such as solubilizers, isotonic agents, suspending agents, emulsifying agents, stabilizers and preservatives.

In some embodiments, an active agent is utilized in an aerosol formulation to be administered via inhalation. As examples, the agent is formulated into a pressurized acceptable propellant such as dichlorodifluoromethane, propane and nitrogen.

In some embodiments, an active agent is made into a suppository by mixing with a base, such as an emulsifying base or water-soluble base. In some instances, an active agent is administered rectally via a suppository. The suppository may include vehicles such as cocoa butter, carbowaxes and polyethylene glycols, which melt at body temperature, yet are solidified at room temperature.

In some embodiments, an active agent is formulation in an injectable composition. Typically, injectable compositions are prepared as liquid solutions or suspensions. In some instances, a solid form is provided which is suitable for solubilization or suspension in a liquid vehicle prior to injection. In some embodiments, an active agent is emulsified or the active agent is encapsulated in a liposome vehicle.

In some embodiments, unit dosage forms for oral or rectal administration, such as syrups, elixirs and suspensions are provided wherein each dosage unit (e.g., teaspoonful, tablespoonful, table, suppository) comprises a predetermined amount of the composition comprising one or more active agents. In some embodiments, unit dosage forms for injection or intravenous administration comprises the active agent in a composition as a solution in sterile water, normal saline or other pharmaceutically acceptable carrier.

In some embodiments, an active agent is formulated for delivery by a continuous or controlled delivery system. Examples include the use of continuous or controlled delivery devices in combination with catheters, injection devices and the like. In some embodiments, the composition is delivered using a pump, including mechanical and electromechanical infusion pumps. In general, pumps provide consistent and/or controlled release of the composition over time. In some embodiments, the active agent is in a liquid formulation in a drug-impermeable reservoir, and is delivered in a continuous or controlled manner to a patient. In some embodiments, a drug delivery system is at least partially implantable. An implantable device can be implanted at any suitable implantation site using methods and devices well known in the art. Implantation sites include, but are not limited to, a subdermal, subcutaneous, intramuscular or other suitable site within a subject's body. Subcutaneous implantation sites are used in some embodiments for convenience in implantation and removal of the drug delivery device. In some embodiments, the active agent is delivered in a controlled release system. In exemplary embodiments, the active agent is administered using intravenous infusion, implantable osmotic pump, transdermal patch or liposomes.

In some embodiments, an active agent of a composition described herein is formulated into absorptive materials, such as sutures, bandages and gauze; or coated onto the surface of solid phase materials, such as surgical staples, zippers and catheters to deliver the agent.

### Dosing and Treatment Regimens

The compositions and agents described herein may be administered to a patient in one or more doses. In some embodiments, a composition comprises one or more active agents. In cases wherein a composition comprises two active agents, a patient may be administered one dose of one active agent and another dose of the other active agent. In some embodiments, an active agent is an agent that inhibits viral binding to a host cell protein. In some embodiments, an active agent is a tyrosine kinase inhibitor. In some embodiments, an active agent is administered an amount from about 1 mg to about 1,000 mg, from about 1 mg to about 950 mg, from about 1 mg to about 900 mg, from about 1 mg to about 850 mg, from about 1 mg to about 800 mg, from about 1 mg to about 750 mg, from about 1 mg to about 700 mg, from about 1 mg to about 650 mg, from about 1 mg to about 600 mg, from about 1 mg to about 550 mg, from about 1 mg to about 500 mg, from about 1 mg to about 450 mg, from about 1 mg to about 400 mg, from about 1 mg to about 350 mg, from about 1 mg to about 300 mg, from about 1 mg to about 250 mg, from about 1 mg to about 200 mg, from about 1 mg to about 150 mg, from about 1 mg to about 100 mg or from about 1 mg to about 50 mg per dose. In some embodiments, an active agent is administered an amount from about 10 mg to about 1,000 mg, from about 10 mg to about 950 mg, from about 10 mg to about 900 mg, from about 10 mg to about 850 mg, from about 10 mg to about 800 mg, from about 10 mg to about 750 mg, from about 10 mg to about 700 mg, from about 10 mg to about 650 mg, from about 10 mg to about 600 mg, from about 10 mg to about 550 mg, from about 10 mg to about 500 mg, from about 10 mg to about 450 mg, from about 10 mg to about 400 mg, from about 10 mg to about 350 mg, from about 10 mg to about 300 mg, from about 10 mg to about 250 mg, from about 10 mg to about 200 mg, from about 10 mg to about 150 mg, from about 10 mg to about 100 mg or from about 10 mg to about 50 mg per dose. In some embodiments, an active agent is administered an amount from about 25 mg to about 1,000 mg, from about 25 mg to about 950 mg, from about 25 mg to about 900 mg, from about 25 mg to about 850 mg, from about 25 mg to about 800 mg, from about 25 mg to about 750 mg, from about 25 mg to about 700 mg, from about 25 mg to about 650 mg, from about 25 mg to about 600 mg, from about 25 mg to about 550 mg, from about 25 mg to about 500 mg, from about 25 mg to about 450 mg, from about 25 mg to about 400 mg, from about 25 mg to about 350 mg, from about 25 mg to about 300 mg, from about 25 mg to about 250 mg, from about 25 mg to about 200 mg, from about 25 mg to about 150 mg, from about 25 mg to about 100 mg or from about 25 mg to about 50 mg per dose.

In some embodiments, an active agent is administered an amount from about 25 mg to about 100 mg per dose. In some embodiments, an active agent is administered an amount from about 100 mg to about 200 mg per dose. In some embodiments, an active agent is administered an amount from about 200 mg to about 400 mg per dose. In some embodiments, an active agent is administered an amount from about 400 mg to about 500 mg. In some embodiments, an active agent is administered an amount from about 500 mg to about 1,500 mg.

In some embodiments, an active agent is administered in about a 50 mg dosage. In some embodiments, an active agent is administered in about a 150 mg dosage. In some embodiments, an active agent is administered in about a 300 mg dosage. In some embodiments, an active agent is administered in about a 450 mg dosage. In some embodiments, an active agent is administered in about a 900 mg dosage.

A composition as described herein, in various embodiments, comprises two active agents. In some embodiments, one active agent is a tyrosine kinase inhibitor. In some embodiments, two active agents are tyrosine kinase inhibitors. In some embodiments, a tyrosine kinase inhibitor is erlotinib. In some embodiments, a tyrosine kinase inhibitor is sunitinib. In an exemplary embodiment, the composition comprises erlotinib and sunitinib. In some embodiments, sunitinib is administered as a 50 mg dose. In some embodiments, sunitinib is administered in a 150 mg dose. In some embodiments, sunitinib is administered in a 300 mg dose. In some embodiments, sunitinib is administered in any dosage from about 5 mg to about 500 mg, from about 5 mg to about 450 mg, from about 5 mg to about 400 mg, from about 5 mg to about 350 mg, from about 5 mg to about 300 mg, from about 5 mg to about 250 mg, from about 5 mg to about 200 mg, from about 20 mg to about 500 mg, from about 20 mg to about 450 mg, from about 20 mg to about 400 mg, from about 20 mg to about 350 mg, from about 20 mg to about 300 mg, from about 20 mg to about 250 mg, or from about 20 to about 200 mg. In some embodiments, erlotinib is administered as a 150 mg dose. In some embodiments, erlotinib is administered in a 450 mg dose. In some embodiments, erlotinib is administered as a 900 mg dose. In some embodiments, erlotinib is administered in any dosage from about 30 mg to about 1,000 mg, from about 30 mg to about 950 mg, from about 30 mg to about 900 mg, from about 30 mg to about 850 mg, from about 30 mg to about 800 mg, from about 30 mg to about 750 mg, from about 30 mg to about 700 mg, from about 30 mg to about 650 mg, from about 30 mg to about 600 mg, from about 30 mg to about 550 mg, from about 30 mg to about 500 mg, from about 30 mg to about 450 mg, from about 30 mg to about 400 mg, from about 30 mg to about 350 mg, from about 30 mg to about 300 mg, from about 30 mg to about 250 mg, from about 30 mg to about 200 mg, from about 30 mg to about 150 mg or from about 30 mg to about 100 mg.

In various implementations, a composition is administered to a patient diagnosed with an infection caused by an enveloped virus, a patient suspected to have an infection caused by an enveloped virus or a patient susceptible to contract an infection caused by an envelope virus. In an exemplary embodiment, the enveloped virus is from the family Filoviridae. In an exemplary embodiment, the enveloped virus is Ebola virus. In some embodiments, the composition comprises a combination of tyrosine kinase inhibitors. In some embodiments, the composition comprises erlotinib and sunitinib. In some embodiments, the patient is administered from about 1 mg to about 100 mg sunitinib and from about 100 mg to about 200 mg erlotinib. In some embodiments, the patient is administered from about 100 mg to about 200 mg sunitinib and from about 100 to about 200 mg erlotinib. In some embodiments, the patient is administered from about 100 mg to about 200 mg sunitinib and from about 400 mg to about 500 mg erlotinib. In some embodiments, the patient is administered from about 200 mg to about 400 mg sunitinib and from about 800 to about 1,000 mg erlotinib. In some embodiments, the patient is administered about 50 mg of sunitinib and about 150 mg of erlotinib. In some embodiments, the patient is administered about 150 mg of sunitinib and about 150 mg or erlotinib. In some embodiments, the patient is administered about 150 mg of sunitinib and about 450 mg of erlotinib. In some embodiments, the patient is administered about 300 mg of sunitinib and about 900 mg of erlotinib. In some instances, the active agents are administered together. In some instances, the active agents are administered separately.

In many implementations of the disclosure, the amount of active agent per dose is determined on a per body weight basis. For example, in an embodiment, the active agent is administered in an amount of about 0.5 mg/kg to about 100 mg/kg, including, from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, and from about 90 mg/kg to about 100 mg/kg. Those of skill will readily appreciate that dose levels often vary as a function of the specific active agent administered, the severity of the symptoms of an infected patient and the susceptibility of the subject to side effects. Preferred dosages for a given compound are readily determinable by those of skill in the art.

The dose of an active agent in a composition described may be administered multiple times. The frequency of administration, in some instances, is dependent on the method of use, for example, for treatment of an infection caused by an enveloped virus or inhibition of infection by an enveloped virus. In some embodiments, an active agent is administered once per month, twice per month, three times per month, every other week, once per week, twice per week, three times per week, four times per week, five times per week, six times per week, every other day, daily, twice a day, three times a day or four times a day. As previously described, an active agent may be administered continuously.

The duration of administration of the active agent (period of time over which the agent is administered), in many instances, varies depending on a number of factors. Examples of such factors include, without limitation, patient response, severity of symptoms, viral load and virus type (e.g., Ebola virus versus Corona virus). In an example, an active agent is administered over a period of time of about one day to about one week, about one week to about two weeks, about two weeks to about four weeks, about one month to about two months, about two months to about four months, about four months to about six months, about six months to about eight months, about eight months to about 1 year, about 1 year to about 2 years or more.

The compositions provided herein are suitable for administration to a subject using any available method and route appropriate for drug delivery, including in vivo and ex vivo methods, as well as systemic and localized routes of administration. Routes of administration include, without limitation, intranasal, intramuscular, intratracheal, subcutaneous, intradermal, topical, intravenous, rectal, nasal, oral and any other enteral and parenteral routes of administration. In some instances, routes of administration are combined and/or adjusted depending on the active agent(s) and/or the desired effect. In some embodiments, a composition comprises two active agents, with each agent being administered to a patient using different routes. Parenteral routes include, without limitation, topical, transdermal, subcutaneous, intramuscular, intraorbital, intracapsular, intraspinal, intrasternal, intravenous routes, and any route of administration other than through the alimentary canal. Parenteral administration can be conducted to effect systemic or local delivery of the active agent. Where systemic delivery is desired, administration may involve invasive or systemically absorbed topical or mucosal administration of pharmaceutical preparations. Enteral routes of administration include, but are not limited to, oral and rectal (e.g., by use of suppository) delivery. In some embodiments, the composition is administered through the skin or mucosa by, for example, topical application, transdermal transmission, injection or epidermal administration. For transdermal transmission, absorption promoters or iontophoresis are suitable methods. Iontophoretic transmission may be accomplished using commercially available patches that deliver a product continuously via electric pulses through unbroken skin over periods of several days or more.

In some embodiments, an active agent of a composition described herein inhibits binding between an Ebola viral protein and host proteins. Host proteins may include, but are not limited to, μ subunits of clathrin adaptor protein complexes, e.g., AP1, AP2, AP3, AP4, AP5, AP2M1/2, AP1M1/2, AP3M1, AP4M1, AP5M1. In some embodiments, the active agent inhibits host protein kinase activity of kinases that modulate the activity of host proteins. In some embodiments, the active agent inhibits GAK (cyclin G-associated kinase). In some embodiments, the active agent inhibits AAK1 (adaptor-associated kinase 1). Active agents which inhibit AAK1 include, but are not limited to, erlotinib, sunitinib, and PKC-412. In addition to treating Filoviridae, Togaviridae, Bunyaviridae, Coronaviridae and/or Arenaviridae, the active agent may be administered to treat subjects co-infected with one or more additional clathrin adaptor protein binding viruses. Examples of clathrin adaptor protein binding viruses include, but are not limited to, HCV, HIV, yellow fever virus (YFV); Dengue virus, including Dengue types 1-4; Japanese Encephalitis virus; Murray Valley Encephalitis virus; St. Louis Encephalitis virus; West Nile virus; tick-borne encephalitis virus; Hepatitis C virus (HCV); Kunjin virus; Central European encephalitis virus; Russian spring-summer encephalitis virus; Powassan virus; Kyasanur Forest disease virus; Ilheus virus; Apoi virus; GB virus A and B; Louping ill virus and Omsk hemorrhagic fever virus.

In some embodiments, a composition comprising an active agent (e.g., tyrosine kinase inhibitor) or combination of active agents, when administered to a patient infected with an enveloped virus (e.g., Ebola virus), reduces the amount of infectious viral particles produced by the virus by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, compared to the number of infectious viral particles produced by the cell not contacted with the composition.

In some embodiments, an active agent or combination of active agents inhibit binding of a viral protein (e.g., Ebola virus protein) to a host protein (e.g., clathrin adaptor protein such as AP2M1 or other μ subunits of clathrin AP complexes) with a 50% inhibitory concentration (IC₅₀) of about 100 μm to about 50 μm, from about 50 μm to about 25 μm, from about 25 μm to about 10 μm, from about 10 μm to about 5 μm, from about 5 μm to about 1 μm, from about 1 μm to about 500 nM, from about 500 nm to about 400 nm, from about 400 to about 300 nm, from about 300 nm to about 250 nm, from about 250 nm to about 200 nm, from about 200 nm to about 150 nm, from about 150 to about 100 nm, from about 100 to about 50 nm, from about 50 nm to about 30 nm, from about 30 nm to about 25 nm, from about 25 nm to about 20 nm, from about 20 nm to about 15 nm, from about 15 nm to about 10 nm, from about 10 nm to about 5 nm or less than about 5 nm.

In some embodiments, an active agent or combination of active agents inhibits viral replication of an enveloped virus (e.g., Ebola virus) by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the level of viral replication in the absence of the therapeutic. In an embodiment, an effective amount of an active agent or a combination of active agents is an amount that, when administered in one or more doses to a host infected (e.g., diagnosed) with an enveloped virus (e.g., Ebola virus), reduces viral load in the subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, compared to the viral load in the subject not treated with the active agent. In some embodiments, an active agent or combination of active agents inhibits binding of a viral protein (e.g., Ebola viral protein) to a host clathrin adaptor protein by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more.

In some embodiments, viral load is measured by measuring the titer or level of virus in a biological sample of a subject. Biological samples include, without limitation, blood, serum, tears, sweat, saliva and urine. In some embodiments, the viral load is obtained by quantifying a viral nucleic acid sequence in a subject. In some embodiments, viral load of a subject is compared to the viral load of a reference sample. In some embodiments, the reference sample is obtained from a patient infected with a virus, for example, Ebola virus. Methods for measuring viral load include, but are not limited to, quantitative polymerase chain reaction and branched DNA test.

In certain embodiments, viral load is predictive of response to treatment if the viral load at a first time point is different in subjects that respond to treatment compared to subjects that do not respond to treatment. It will be understood that a variety of statistical analysis can be performed to identify a statistically significant association between viral load and response of the subject to the treatment. In some embodiments, the viral load is elevated in subjects that will not respond to a treatment. In some embodiments, the viral load is decreased in subjects that will not respond to treatment. In some embodiments, the sequence of the virus, e.g., serotype, in certain examples, is different in subjects that will not respond to treatment. Furthermore, viral load may predict a level of response to treatment, for example partial or temporary response to treatment versus a full response.

In some embodiments, provided herein is a method for predicting response to treatment for Ebola virus, including providing a biological sample from a subject infected with Ebola virus, amplifying Ebola viral nucleic acids in a nucleic acid amplification assay, and detecting the amplified nucleic acids. The method may further involve detecting the viral load of the subject. The method may further involve determining a score based on a scoring matrix.

Time points for monitoring and response-to-treatment methods include any interval of time. In some embodiments, the time points are 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years or longer apart.

Methods provided herein which treat and/or inhibit infection by an enveloped virus have may be combined with methods for diagnosing, quantitating, serotyping and prognosing viral infection (e.g., Ebola viral infection), as well as monitoring a response of a subject with viral infection to treatment. In some embodiments, these methods are used to calculate viral load. In some embodiments, these methods identify subjects as susceptible for viral treatment. In some embodiments, the progression of viral infection in a subject is monitored. For example, by comparing the quantity of viral nucleic acids prior to treatment with the quantity of viral nucleic acids after treatment.

### Kits

Provided herein, in one aspect, are kits which include one or more reagents or devices for the performance of the methods disclosed herein. In some embodiments, the kit comprises a composition as described herein. In some embodiments, the kit comprises one, two or more active agents. In some embodiments, the active agents are formulated together. In some embodiments, the active agents are formulated separately. In some embodiments, the kit comprises a means to administrate a composition comprising one or more active agents as described herein. In some embodiments, a kit comprises a therapeutically effective amount of an agent that inhibits binding of an enveloped virus to a host cell protein. In some embodiments, a kit comprises a therapeutically effective amount of a tyrosine kinase inhibitor. In some embodiments, a kit comprises erlotinib, or a salt or solvate of erlotinib. In some embodiments, a kit comprises sunitinib, or a salt or solvate of sunitinib. In some embodiments, a kit comprises erlotinib or a salt or solvate of erlotinib and sunitinib or a salt or solvate of sunitinib.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

In some embodiments, many reagents may be provided in a kit, only some of which should be used together in a particular reaction or procedure.

In some embodiments, a kit provided herein includes a carrier means being compartmentalized to receive in close confinement one or more containers such as vials, tubes, and the like, each of the containers comprising one of the separate elements to be used in a method provided herein.

### EXAMPLES

The following examples are set forth to illustrate more clearly the principle and practice of embodiments disclosed herein to those skilled in the art and are not to be construed as limiting the scope of any claimed embodiments.

### Example 1

### Knockdown of AAK1 and GAK Reduces Ebola Entry

To identify a role of AAK1 and GAK in Ebola entry, a viral entry assay was performed using AAK1 and GAK knockdown Vero cells.

Vero cells were transfected with two independent siRNAs against AAK1 and GAK or a non-targeting (NT) control using silmporter transfection reagent (Millipore) according to manufacturer's instructions. At 48 hours post transfection, cells were infected with recombinant, GFP-tagged vesicular stomatitis virus expressing EBOV glycoprotein (rVSV-EBOV-GP) for 4 hours. Knockdown of AAK1 and GAK in the respective samples was confirmed by quantitative real-time PCR. At 20 hours post infection, the percent of GFP positive cells out of the total live cell population was quantified via flow cytometry as a measure of viral entry.

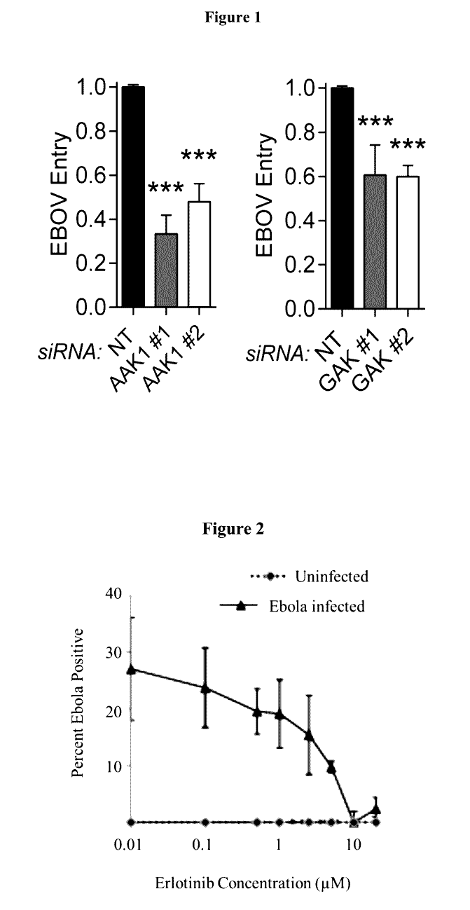

The percentage of viral entry as quantified by flow cytometry for each knockdown and NT control was plotted in the graph of FIG. 1. Knockdown of AAK1 and GAK resulted in a significant reduction of viral entry as compared to NT control. The results were consistent across both independent siRNAs for each target.

### Example 2

### Erlotinib Inhibits Ebola Virus Infection

To identify an effect of erlotinib on Ebola viral entry, a viral entry assay was performed on Vero E6 cells treated with erlotinib.

Cells: Vero E6 cells were seeded into black-walled, clear-bottom 96-well plates. Cells were infected upon reaching about 90% confluency.

Erlotinib preparation: Drug dilutions were prepared as necessary. Briefly, a 10 mM stock of erlotinib was prepared in 100% DMSO. From the 10 mM stock, a 500 μM stock (working stock) was prepared in water, by diluting 1:20, and subsequently filtered. The working stock was aliquoted and stored frozen at-20° C. Prior to treating cells, the erlotinib stock was sonicated at room temperature for about 20 minutes to improve solubility. The following concentrations of erlotinib were tested in the assay: 0.1 μM, 0.5 μM, 1 μM, 2.5 μM, 5 μM, 10 μM and 20 μM. Cells were treated in the presence of media comprising 10% FBS.

Cell culture media was replaced with erlotinib containing media about 1 hour prior to infection.

Infection: Virus was diluted in serum-free media to an MOI of 0.1. Media was removed from the wells and replaced with 100 μl of virus solution. The wells were incubated for 1 hr at 37° C. with intermittent rocking. Following the 1 hr incubation, the solution was removed from the wells, the wells were washed twice with sterile PBS, and the wells were replaced with 200 μl complete media containing erlotinib. The cells were subsequently incubated. Following 24 hours of incubation, media was replaced with fresh media comprising erlotinib and the cells subsequently incubated.

Cell viability: Cell viability was assessed using an alamarBlue-based assay which briefly includes adding fresh medium with 10% AB to the cells, incubating for 1.5 hours and measuring fluorescence.

Cell fixation: Media was removed from the wells and the plate completely submerged in formalin. The plate was placed at 4° C. for a minimum of 24 hours. After at least 24 hours, the plates were removed from 4° C. and brought to room temperature. The formalin was replaced with fresh formalin. The plate was washed with PBS at least twice. 100 μl of blocking buffer (1% BSA or cell staining buffer) was added to wells and the plate incubated at 4° C. overnight. The wells were then washed with PBS and incubated with 50 μl/well primary antibody (anti-Ebola GP) diluted in 1% BSA for 20 minutes at room temperature on a shaker. After primary antibody incubation, the plate was washed three times with 100 μl PBS. The plate was then incubated on a shaker for 20 min at room temperature with secondary antibody in 1% BSA. After secondary incubation, the plate was washed three times with 100 μl PBS. HCS cell mask deep red was applied to the wells at 1:10,000 dilution in PBS, 100 μl/well. HOX stain was added at 1 drop/ml. The plate was covered in foil and incubated at 4° C. overnight. The plate was then imaged.

The percentage of Ebola positive cells versus concentration of erlotinib was plotted in the graph shown in FIG. 2. FIG. 2 illustrates inhibition of Ebola by erlotinib in a dose-dependent manner.

### Example 3

### Sunitinib and Erlotinib Inhibit Ebola Virus Entry

To identify the effects of sunitinib and erlotinib on Ebola viral entry, a viral entry assay was performed on Vero E6 cells pre-treated with erlotinib, sunitinib or a combination of erlotinib and sunitinib.

Vero cells were pre-treated with varying concentrations of sunitinib, erlotinib, sunitinib and erlotinib, or DMSO vehicle control for 1 hr and subsequently infected with rVSV-EBOV-GP for 4 hours. Virus and inhibitors were washed out, fresh cell media was replenished, and Ebola entry measured via flow cytometry at 20 hours post infection. Combination data was analyzed by MacSynergy.

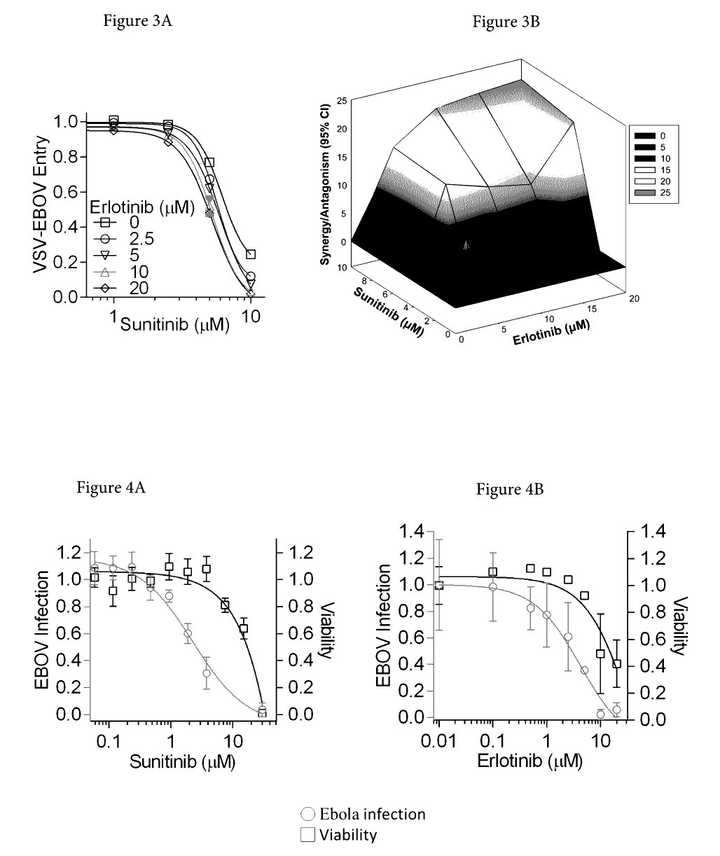

Treatment with sunitinib and erlotinib reduced Ebola entry in a dose-dependent manner, as shown in the graph of FIG. 3A. Sunitinib potently inhibited viral entry with an IC₅₀ of 6.15 μM. The combination of sunitinib and erlotinib displayed measurable synergy without antagonism, as shown in the graph of FIG. 3B. Log volume for the synergy was 25.7. Treatment had no apparent effect on cellular toxicity, as measured by alamarBlue-based assays.

### Example 4

### Sunitinib and Erlotinib Inhibit Ebola Infection

To identify the effects of sunitinib and erlotinib on Ebola replication, a viral entry assay was performed using Vero E6 cells pre-treated with erlotinib or sunitinib.

Vero cells were pre-treated with erlotinib or sunitinib for 1 hr and then infected with rVSV-EBOV-GP in the presence of the erlotinib or sunitinib for 3 hrs under BSL-4 containment. Cells were fixed at 48 hours post infection, and infected cells were quantified with EBOV-GP specific monoclonal antibody and a fluorescently labeled secondary antibody. Cells were counter-stained with Hoescht dye to calculate the fraction of infected cells for the total cell population. Viability was measured by an alamarBlue-based assay.

The effect of various concentrations of sunitinib and erlotinib on overall Ebola infection and cell viability was plotted in the graphs of FIG. 4A (sunitinib) and FIG. 4B (erlotinib). Sunitinib and erlotinib treatment resulted in a dose-dependent reduction in the overall infection with Ebola virus. The EC₅₀ values were calculated to be 2.25 μM for sunitinib and 4.22 μM for erlotinib.

### Example 5

### Erlotinib and Sunitinib for the Treatment of Ebola Virus in Infected Mice

Composition (drug) preparation

Sunitinib is dissolved at 75 mg/ml in DMSO and then further diluted with PBS to a working concentration dependent on the weight of the mouse. 100 μl of the diluted sunitinib is delivered per injection per animal. Sunitinib generally is sonicated to ensure that it is completely dissolved in DMSO, which usually takes about 45 minutes. Erlotinib is dissolved in 6% Captisol at 10 mg/ml. The solution is sonicated for about 10 minutes and then diluted further with 6% Captisol to a working concentration dependent on the weight of the mouse. Aliquots of each drug are stored at −20° C. A starting combination treatment is 30 mg/kg sunitinib and 30 mg/kg erlotinib.

Infection

Day 1: The mice are weighed and pre-treated about 6 hours prior to inoculation. Each drug, combination of drugs and/or vehicle(s) is injected into the mice in separate injections. Generally, for mice treated with a combination of drugs, one drug is injected on the left and another drug is injected on the right side of the mouse body. The treated mice are inoculated with 100 μl virus retro-orbitally by applying proparacaine to the eye to be used for viral inoculation. The mice are anesthetized using isoflurane and monitored to ensure recover from anesthesia.

Day 2: The mice are weighed and their health scored 24 hours post inoculation. The mice are treated with the drug(s) and/or vehicle(s) IP in two separate injections.

Day 3: The mice are weighed and their health scored 48 hours post inoculation. Proparacaine is applied to the eye to be bled. The mice are anesthetized with isoflurance. A retro-orbital bleed is performed to collect about 50 to about 100 μl of blood to measure viral load in the serum. The mice are then immediately treated with the drug(s) and/or vehicles(s) IP in two separate injections. If desired, fluids are administered to the mice to replenish blood loss.

Day 4 and beyond: Administration of drug(s) and/or vehicle(s) continues for a total of five treatments. The mice are continually monitored for weight, health score and survival. Eye ointment (generic ophthalmic ointment with Bacitracin zinc, Neomycin, and polymyxin B antibiotics) is administered if there are signs of infection or proptosis.

Arms

Vehicle only—Captisol and PBS in two injections

Sunitinib—30 mg/kg

Erlotinib—30 mg/kg

Sunitinib (30 mg/kg) and erlotinib (30 mg/kg)

Mice are treated for 10 days and followed for 14 days. The number of mice is dependent on the model. In one experiment, 8 are used per arm.

Results

The percentage survival for mice treated with vehicle only, erlotinib (30 mg/kg), sunitinib (30 mg/kg) or a combination of erlotinib (30 mg/kg) and sunitinib (30 mg/kg) is monitored for 28 days post infection. In this example, 100% of mice treated with erlotinib survive 28 days post infection with Ebola virus.

What is claimed is:

1. A method of treating Filoviridae, Togaviridae, Bunyaviridae, Coronaviridae, or Arenaviridae, the method comprising administering to a patient in need thereof an effective amount of an agent that inhibits binding of the virus to a host μ (mu) subunit of clathrin adaptor protein complexes.

2. The method of claim 1, provided that the μ (mu) subunit is selected from μ subunits of clathrin AP1, AP2, AP3, AP4, and AP5 complexes, and combinations thereof.

3. The method of claim 2, provided that the μ (mu) subunit is selected from μ subunits of AP2M1/2, AP1M1/2 , AP3M1, AP4M1, and AP5M1, and combinations thereof.

4. The method of claim 1, provided that the agent inhibits AAK1, GAK, or a combination thereof.

5. The method of claim 1, provided that the agent comprises a tyrosine kinase inhibitor.

6. The method of claim 1, provided that the agent comprises erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate, or combination thereof.

7. The method of claim 1, provided that the patient is infected with a Filoviridae virus or is susceptible for infection with a Filoviridae virus.

8. The method of claim 7, provided that the patient is infected with Ebola virus or is susceptible for infection with Ebola virus.

9. The method of claim 6, provided that the agent comprises sunitinib, or a salt or solvate thereof, and erlotinib, or a salt or solvate thereof.

10. The method of claim 1, further comprising administering to the patient in need thereof an effective amount of an anticancer agent, an antiviral agent, or a combination thereof.

11. A method of treating Filoviridae, Togaviridae, Bunyaviridae, Coronaviridae or Arenaviridae, the method comprising administering to a patient in need thereof an effective amount of a tyrosine kinase inhibitor.

12. The method of claim 11, provided that the tyrosine kinase inhibitor comprises an epidermal growth factor receptor inhibitor, a platelet-derived growth factor receptor inhibitor, a vascular endothelial growth factor receptor inhibitor, or a combination thereof.

13. The method of claim 11, provided that the tyrosine kinase inhibitor comprises erlotinib, sunitinib, gefitinib, dasatinib, imatinib, lapatinib, imatinib, vandetanib, afatinib, neratinib, axitinib, masitinib, pazopanib, toceranib, lestaurtinib, cediranib, nintedanib, regorafenib, semaxanib, cabozantinib, sorafenib, or a salt, solvate or combination thereof.

14. The method of claim 11, provided that the patient is infected with a Filoviridae virus or is susceptible for infection with a Filoviridae virus.

15. The method of claim 14, provided that the patient is infected with Ebola virus or is susceptible for infection with Ebola virus.

16. The method of claim 11, provided that the tyrosine kinase inhibitor comprises erlotinib, or a salt or solvate thereof, and sunitinib, or a salt or solvate thereof.

17. The method of claim 11, further comprising administering to the patient in need thereof an effective amount of an anticancer agent, an antiviral agent, or a combination thereof.

18. A composition for treating Filoviridae, Togaviridae, Bunyaviridae, Coronaviridae, or Arenaviridae, the composition comprising sunitinib, or a salt or solvate thereof, and erlotinib, or a salt or solvate thereof.

19. The composition of claim 18, provided that the composition is for treating Ebola virus.

20. The composition of claim 18, provided that the composition comprises from about 1 mg to about 1 g of sunitinib, or a salt or solvate thereof, and from about 1 mg to about 1 g of erlotinib, or a salt or solvate thereof.
